(19) United States
(12) Patent Application Publication
(10) Pub. No.: US 2022/0031931 A1
(43) Pub. Date: Feb. 3, 2022

(54) Pressure Switch for Aspiration Systems and Devices

(71) Applicant: Stout Scientific, LLC, Moreno Valley, CA (US)

(72) Inventor: Charles Emmett Stout, Moreno Valley, CA (US)

(21) Appl. No.: 17/393,098

(22) Filed: Aug. 3, 2021

Related U.S. Application Data
(60) Provisional application No. 63/060,544, filed on Aug. 3, 2020.

(51) Int. Cl. A61M 1/00; A61B 17/22

(52) U.S. Cl. CPC A61M 1/75; A61B 17/22; A61B 2017/22079; A61M 1/743; A61M 2210/0693; A61M 1/76

(57) ABSTRACT

Pressure regulations allow for example, tension control on spring valves to regulate cycling behavior for aspiration catheter systems. Unique brain access and procedures are driven by the improved cycling patterns and managed periodic frequencies.

### REFERENCE TO PRIORITY APPLICATION

This application claims the full Paris Convention benefit of, and priority to, U.S. Provisional Patent Application Ser. No. 63/060,544, filed Aug. 3, 2020.

### FIELD OF THE INVENTION

The present inventions relate to mechanisms, devices and systems which are used to cycle pressure for the purpose of removing thrombus, among other things.

### BACKGROUND OF THE DISCLOSURES

Known are system to access and treat the brain, each of these patents being incorporated expressly by reference as if fully set further herein along with their references: U.S. Pat. Nos. 10,413,310; 10,383751; 10,251,739; 10,123,803; 10,016,211; 9,901,435; 9,808,359; 9,566,072; 9,387,098; 9,320,523; 9,220,522; 9,198,387; 8,945,172; 8,932,321; 9,554,851; 9,061,118; 8,419,678; 8,322,469; 9,937,326; 8,277,374; 7,645,275; 7,578,787; 7,326,225 and 6,719,684. Not known are systems which use varied pressure in terms of the periodic frequency to improve outcomes including aspiration in the brain, although sorely needed.

### OBJECTS AND SUMMARY OF THE INVENTION

Briefly stated, pressure regulations allow for example, tension control on spring valves to regulate cycling behavior for aspiration catheter systems. Unique brain access and procedures are driven by the improved cycling patterns and managed periodic frequencies.

According to features of the present invention, there is disclosed an improved pressure switch for cycling pressure between atmospheric and pump pressure, which generates a novel cycle pattern.

According to features of the present invention, there is disclosed a medical device system for cycling pressure between pump pressure and atmospheric pressure, comprising, in combination, at least an aspiration pump, suction tubing connecting the pump to a device, and an associated aspiration catheter, whereby a novel cycle pattern is generated.

According to features of the present invention, there is disclosed an advanced pressure switching mechanism for managing periodic frequency of pressure used during interventional and neurovascular procedures.

### BRIEF DESCRIPTION OF THE DRAWINGS

Various preferred embodiments are described herein with references to the drawings in which merely illustrative views are offered for consideration, whereby:

FIG. 1 shows the prior art's management of the cycle pressure between pump, pressure and atmospheric pressure, where the curve represents decrease in standard method pressure over time;

FIG. 2 shows the curve with the Novel Enhanced Aspiration Device, Systems, Methods, and Products thereby;

FIG. 3 shows an embodiment of the present invention;

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially-feasible embodiment are often not depicted in order to facilitate a less obstructed view of these various embodiments of the present invention.

### DETAILED DESCRIPTIONS

The present inventor has discovered and invented novel and enhanced ways to cycle pressure between pump pressure and standard pressure. Numerous issues exist which make periodic pressure management helpful with aspiration of thrombus and many other activities.

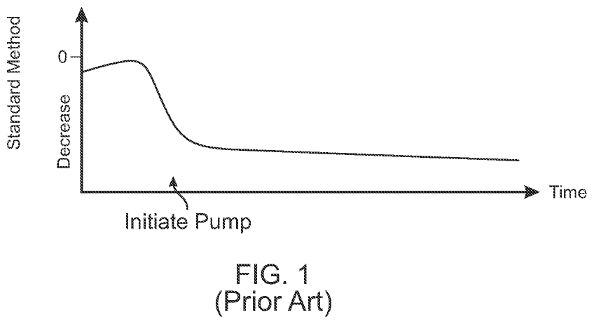

Referring now to FIGS. 1-3, the prior art purpose/function of cycling pressure is shown by FIG. 1, such as known to those skilled in the art. Standard aspiration techniques would benefit from variated and periodic pressure changes, it is respectfully proposed, particularly in the cerebral vasculature, as proposed below and claimed herein and thereafter. In particular, it is known that throbs and emboli are sticky and difficult to apply direct pressure to within the brain particularly.

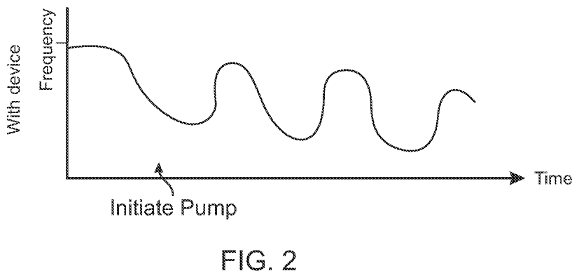

FIG. 2 shows the improved periodic frequency according to the present invention; and it shows, it is respectfully proposed, that once pump is initiated that sinusoidal for example or other periodic frequency changes can support more effective removal of thrombus over time. Those of skill in the art understand that different pressures facilitate the dislodgment and related management of thrombus and other foreign bodies as well in such situations.

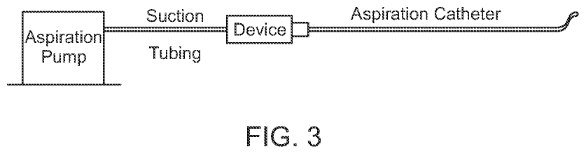

FIG. 3 shows an exemplary device, according to the instant teachings, namely One placement of the pressure switch (labeled DEVICE) between the suction tubing and aspiration catheter. Alternate placement likewise is contemplated according to the instant teachings, along with other devices (stent retrievers, flow diverters, coils and the like devices) and therapies and procedures.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment,” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled. steps are indicative of one embodiment of the presented method. Other steps and methods may be conceived that are equivalent function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed—some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using, consisting of, or consisting essentially of language. When used in the claims, whether as filed or added per amendment, the transition term “consisting of” excludes any element, step, or ingredient not specified in the claims. The transition term “consisting of” essentially limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O interface may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antennae, and a network interface device (e.g., a network interface card (NIC), Wi-Fi card, cellular modem, data jack, Ethernet port, modem Jack, HDMI port, mini-HDMI port, USB port), touchscreen (e.g., CRT, LCD, LED, AMOLED, Super AMOLED), pointing device, trackpad light (e.g., LED), light/image projection device, or a combination thereof.

Memory according to the invention refers to a non-transitory memory, which is provided by one or more tangible devices which preferably include one or more machine readable medium on which is stored one or more sets of instructions (e.g., software) embodying any one or more of the methodologies or functions described herein. The software may also reside, completely or at least partially, within the main memory processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices, (e.g., SD, micro-SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An improved pressure switch for cycling pressure between atmospheric and pump pressures which generates and communicates a novel cycle pattern.

2. The improved pressure switch of claim 1, whereby a curve which defines the cycle pattern shows that induced variations of cyclic pressure generate alternate periodic frequencies, for example, fixed or unfixed sinusoidal patterns.

3. The improved pressure switch of claim 2, effective for use in neurovascular and interventional procedures with currently existing medical devices.

4. The improved pressure switch of claim 3, effective for use with newly developed or later developed medical devices.

5. A medical device system for cycling pressure between pump pressure and atmospheric pressure comprising, in combination:
at least an aspiration pump, suction tubing connecting the at least a pump to a device; and
an associated aspiration catheter, whereby a novel cycle pattern is generated.

6. The medical device system of claim 5, defined by the curve in FIG. 2.

7. The medical device system of claim 6, whereby a spring-loaded mechanism is effective to actuate a release valve as mechanical means to regulate pressure.

8. The medical device system of claim 7, further comprising tension control on the spring valve.

9. The medical device system of claim 8, wherein cycling behavior is regulated.

10. The medical system of claim 9, forming a kit for using any medical device along with the pump and tubing and aspiration catheter.

11. The medical device system of claim 10, wherein the aspiration catheter is designed for neurovascular use.

12. The medical device system of claim 10, wherein the aspiration catheter is designed for peripheral vascular use.

13. The medical device system of claim 10, using an aspiration catheter or modified device for cardiovascular applications.

14. An advanced pressure switching mechanism for managing periodic frequency of pressure used during interventional and neurovascular procedures.

15. The advanced pressure switching mechanism as defined by claim 14 and FIG. 2, herein, which is expressly incorporated by reference herein.

16. The advanced pressure switching mechanism of claim 15, further comprising a pump, specialized tubing and an aspirating catheter according unique specification for neuromuscular usage.

17. The advanced pressure switching mechanism of claim 16, whereby tension control on spring valves is used to manage cycling behavior for specialized aspiration catheters.

18. The advanced pressure switching mechanism of claim 17, whereby tension control on spring valves is used to managed cycling behavior for generally available aspiration catheters.

19. The advanced pressure switching mechanism of claim 18, whereby catheters and catheter systems may access and aspirate previously challenged vessels and locations in the brain.

20. The advanced pressure switching mechanism of claim 19, useful to access the cerebral septum, among other situses.
